(19) United States
(12) Patent Application Publication
(10) Pub. No.: US 2013/0172994 A1
(43) Pub. Date: Jul. 4, 2013

(54) SUPPORTING AND FORMING TRANSITIONAL MATERIAL FOR USE IN SUPPORTING PROSTHESIS DEVICES, IMPLANTS AND TO PROVIDE STRUCTURE IN A HUMAN BODY

(71) Applicant: Hilton BECKER, Boca Raton, FL (US)

(72) Inventor: Hilton BECKER, Boca Raton, FL (US)

(21) Appl. No.: 13/777,368

(22) Filed: Feb. 26, 2013

Related U.S. Application Data
(63) Continuation-in-part of application No. 12/832,161, filed on Jul. 8, 2010, now abandoned.
(60) Provisional application No. 61/351,062, filed on Jun. 3, 2010.

(51) Int. Cl. A61F 2/12

(52) U.S. Cl. CPC A61F 2/12
USPC 623/8

(57) ABSTRACT

A fabric for use in the human body composed of non-absorbable micro diameter threads contained in absorbable materials, such that over time the absorbable material dissolves or is absorbed by the body and the non-absorbable micro diameter threads cause the body to create a collagen scaffold transferring load from the absorbable material to the collagen scaffold. The fabric can be coated or impregnated with materials to reduce infection, provide tissue growth, reduce scar tissue or other medical purpose. Threads of non-absorbable material can be coated with absorbable material to create a fabric.

### PRIORITY CLAIM

This application is a continuation-in-part of U.S. patent application Ser. No. 12/832,161, filed on Jul. 8, 2010, which claims priority to U.S. Provisional Application No. 61/351,062, filed on Jun. 3, 2010, the disclosure and contents of which are expressly incorporated herein by reference.

### FIELD OF THE INVENTION

This invention relates to a forming and supporting material usable for breast reconstruction following a mastectomy, breast augmentation or modification, or the treatment of breast implant complications, especially capsular contraction. The material may also be utilized in other areas of reconstructive surgery where initial non-yielding, strong support is required or desired to be provided which after a suitable period of time, when healing has occurred, transforms into a flexible matrix having the characteristic of human tissue acting as a scaffold that allows host collagen growth without restriction of elasticity, flexibility and motion.

### BACKGROUND FOR THE INVENTION

Implants and methods for breast reconstruction and augmentation are well known and have been used for over forty years. The two primary difficulties with implants have been the issue of supporting and restraining the implant to maintain its location and to supply an extra layer of tissue over the implant while allowing appropriate movement and preventing fibrous scar tissue encapsulation. The problems of providing appropriate support while maintaining the desired movement have been dealt with in many of the patent applications discussed herein. The greater support provided, the less natural movement is achieved. This problem is amplified by the creation of scar tissue. When a foreign body is implanted into the body, the implant material is walled off by the response of the human tissue. This is commonly referred to as encapsulation. As the capsule that is formed is scar tissue, it is fairly rigid and in certain cases may actually contract, resulting in hardness around the implant. The encapsulation can also lead to the problem of spherical scar contracture. The scar tissue surrounds the prosthetic device and as it contracts causes the configuration of the implant to be altered as well as creating hardness, discomfort, displacement and pain. Implants are traditionally placed under the muscle or partially under the muscle when performing breast reconstruction. This is done to provide an extra layer of tissue over the implant. By providing a material which acts as a scaffold over the implant it would not be necessary to perform additional surgery to elevate the muscle over the implant.

Numerous approaches have been proposed to address these problems. Cronin, U.S. Pat. No. 3,293,663, filed on Dec. 27, 1966, was one of the first implant patents for breast prosthesis that proposed support use of corrugated fabric against the chest wall, which is anchored to the back portion of the implant. The Perras U.S. Pat. No. 3,665,520, filed on May 30, 1972, later proposed support using a Dacron strip affixed to the back wall of the implant. Frank, U.S. Patent Application No. U.S. 2007/0088434A, filed on Nov. 29, 2006, proposed support using a sheet of prosthetic material configured to form a sling-shaped receiving area to support the breast implant.

Popov PCT Application WO2007/004214A2, filed Jun. 28, 2006, uses a basket-shaped structure to provide support to an implant or the mammary gland itself.

These proposals provided support but did not fully allow natural motion, nor did they deal with the problem of encapsulation.

A second approach has been to have the implant surface textured or modified to be bio-compatible to provide support and hopefully reduce capsule formation. This is seen in McGhan, U.S. Pat. No. 6,913,626B2, filed on Jul. 5, 2005, and Agerup, Patent Application. No. 2008/0312739A1, filed on Dec. 18, 2008. There has been only moderately success with such procedures and later studies have indicated it has not achieved the desired objectives.

Another approach has been use of an outer container or pouch to hold the implant prosthesis and interact with the body by in-growth to provide support and try to reduce the occurrence of capsular contracture's inhibiting properties.

The use of sheet or film type materials which are biologically absorbable into the body to provide support has been tried and is disclosed in Naficy, U.S. Pat. No. 4,298,998, filed on Nov. 10, 1981. The use of non-bioabsorbable mesh coated with a bioabsorbable material is seen in Buevich, U.S. Patent App. 2008/0132922A1, filed on Jun. 6, 2008, and using biocompatible but non-bioabsorbable meshes to provide supporting structure is disclosed in Chen, U.S. Patent App. 2009/0082864A1, filed on Mar. 26, 2009, O'Keefe, U.S. Pat. No. 4,936,858, filed on Jun. 26, 1990, and Maxwell, U.S. Patent App. 2009/0125107A1, filed on May 14, 2009. However, the use of these various meshes has not resolved the problem related to breast or other type implants as corrective operation rates to address need for more natural movement and capsular contracture have been unacceptably high.

The use of fabric type materials has been known for some time, the purpose of which has been to provide the function of a permanent re-enforcing structure. These materials have usually incorporated a permanent non-absorbable structure which at times is laced together or coated with various bioabsorbable portions used as filler type material. See Landi, U.S. Pat. No. 5,326,355, filed on Jul. 5, 1994, and Ledergerber, U.S. Pat. No. 4,955,907, filed on Sep. 11, 1990. Typically these fabric type materials contain a weave or structure, usually a knotted mesh that will be of a biocompatible, non-absorbable material which may have a bio-absorbable component. After absorbance of any bio-absorbable component, the remaining structure which is intended to provide needed support provides insufficient strength and in many cases cause scar tissue to form about the permanent, non-absorbable material resulting in the scar tissue formation, discomfort and hardness, as well as need for corrective surgery. The use of mesh to provide additional support and use of textured surfaces to promote tissue growth and affix the implant is taught in co-pending U.S. patent application Ser. No. 12/169,000, filed on Jul. 8, 2008 which is a continuation in part of U.S. patent application Ser. No. 12/026,032, filed on Feb. 5, 2008. Also incorporated by reference are co-pending applications U.S. patent application Ser. No. 12/552,352 filed on Sep. 2, 2009, U.S. patent application Ser. No. 12/556,050, filed on Sep. 9, 2009, and U.S. patent application Ser. No. 12/026,032, filed on Feb. 5, 2008, and co-pending U.S. application Ser. No. 12/832,161, filed Jul. 8, 2010. However, none of these prior art attempts have solved the problem of providing both adequate support, strength, elasticity and scaffold function with acceptable control or elimination of capsular contracture, scar tissue formation, discomfort and hardness.

The prior art did not achieve support with flexibility and motion. This invention allows the body to create its own support structure.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one aspect thereof, comprises a fabric comprising portions which are permanent non-absorbable micro-threads or strands and portions which are absorbable, such that the fabric transitions from a sheet to an array of non-absorbable threads or strands which are not connected to each other. As the absorbable portion dissipates, the implant and tissue are provided structural support by the collagen structure created by the body due to the non-absorbable portions of the fabric. Since the absorbable fabric areas dissipate and provide disruptions to a continuous type of structure, there is much less scar tissue formed and capsular contracture does not become a problem. The material can be conformed into a variety of configurations and reduced to single strands with intermittent absorbable material, can also provide support.

It is the desire of this invention by virtue of its unique and novel approach to provide the following benefits to breast implant and other restorative procedures; provide support but allow natural movement, prevent or reduce to nominal levels capsular contracture, allow use of smooth surfaced gel implant prosthesis more effectively, with placement above the muscle, stop or minimize rippling from occurring, provide treatment to encourage in-growth and against potential infections, and provide the scaffold for the forming of a new tissue layer that is permanent but elastic, simulating natural human supportive tissue.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### DESCRIPTION OF DRAWINGS

FIG. 1 illustrates a sponge-type sheet of absorbable material with non-absorbable threads contained therein.

FIG. 2(a) illustrates a thread woven with absorbable material housing fine strands of non-absorbable material.

FIG. 2(b) illustrates the thread weave of FIG. 2(a) after absorption has occurred and shows the intentional break in non-absorbable fibres.

### DESCRIPTION OF THE PREFERRED EMBODIMENTS OF THE INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

The present invention overcomes the disadvantages of the prior art by incorporating a non-absorbable material that will provide the needed support when a breast implant is being inserted or reconstructive surgery is being performed, yet prevents capsule constriction from occurring, and promotes for tissue collagen growth which is elastic and allows natural movement. Although absorbable mesh fabrics work well initially, once completely absorbed there is no or little residual strength. The mesh initially will function by stimulating fibroblasts to secrete collagen to form a structure of collagen that provides support. This mechanism is a piezo-electric current generated from the surface of the mesh material. Once formed in response to the piezo-electric current, the collagen remains in place while the piezo-electric current continues. Eventually the absorbable mesh dissipates and the piezo electric stops resulting in the body then removing or breaking down the collagen structure created.

As further explained herein, an absorbable fabric which contains non-absorbable microscopic fine permanent fibres, would leave the microscopic fine permanent fibres, would leave the microscopic fine fibres to remain and provide a piezo electric current thereby maintaining the collagen in the area to provide the necessary support.

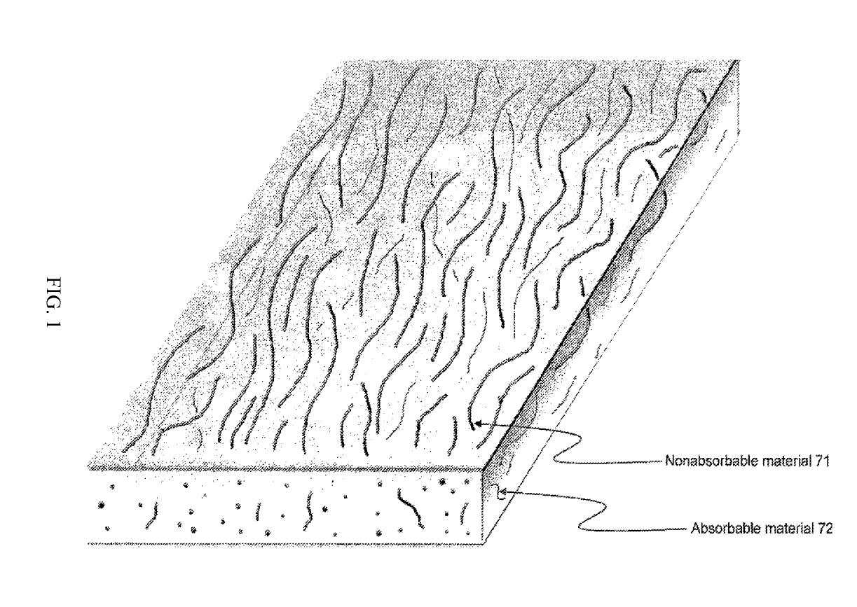

The present invention in a preferred embodiment FIG. 1 shows a fabric comprising portions which are permanent threads or fibres (as used in this application threads and fibres are the same), i.e. non-absorbable 71 and portions which are absorbable 72, such that the fabric transitions from a continuous sheet to an array of non-absorbable threads which are not connected to each other. We call this a transitional mesh since the sheet changes its structural composition. The term “transitional” can also apply to a thread which utilizing this invention will also change its structural composition.

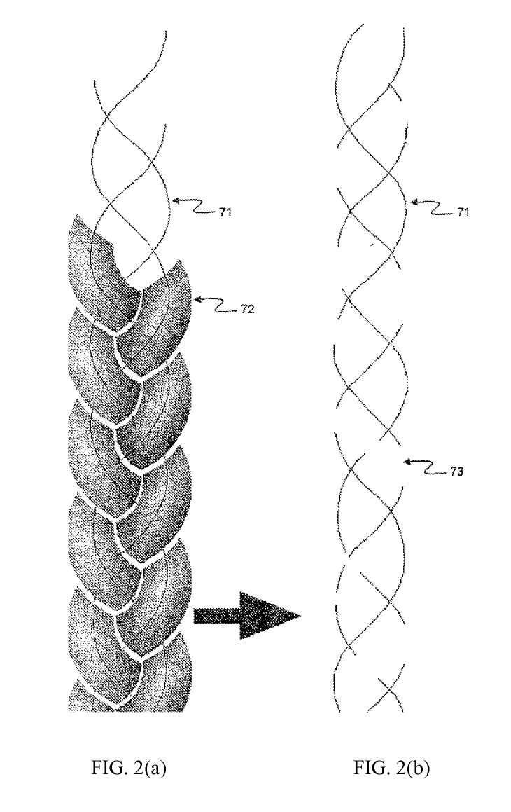

The absorbable material 72 can be, but is not required to be, of equal or greater strength to that of the non-absorbable material, allowing the fabric to provide support to the implant during the healing process. As the absorbable portion dissipates, the implant and tissue are provided structural support by the collagen structure created by the body due to the non-absorbable portions of the fabric 71, which being of a micro diameter, preferably between 30 microns to one tenth of a millimeter, causes the body to create a structural support lattice. The quantity of microfibers would vary depending on the desired support needed. Thus, as the bioabsorbable material dissolves or dissipates, the collagen lattice grows, caused by the continuing irritation of the micro diameter fiber or threads. The result is a natural structure that can move and is flexible giving a natural appearance. FIG. 2(a) shows the absorbable and non-absorbable components of the thread where the thread non-absorbable material is housed within the absorbable material and the strands of non-absorbable material 71 remain after absorption FIG. 2(b). The elasticity, or ability to elongate, of the thread can be accomplished by the type of weave used for the thread and its components, the fibres being discontinuous and having breaks in its continuity 73 either in the thread fiber components or use of an elastic material for the thread component, or any combination of these type or similar constructions. The differing types of weaving or threading, size and type of thread, being monofilament or not, and patterns utilized gives one skilled in the art flexibility to select the type of transitional mesh or thread to meet the specific requirements needed, as long as the non-absorbable thread component provides the needed impetus to have the body create a scaffold structure.

The materials in all of the embodiments may also be impregnated or coated with medical material as infection-fighting antibiotics or drugs to provide pain relief, to facilitate reduction of scar tissue, or to promote tissue growth.

The fabric used for the absorbable and non-absorbable segments can have a varied height of surface which promotes tissue in-growth with the segment and helps impede scar tissue formation.

Although use of the invention has been primarily shown for breast implant utilization, the fabric can be used for other surgical repairs or utilized in areas where strength is needed but natural motion and minimal scar tissue formation is desired.

The types of fabric that can be utilized can vary depending upon the needs and desires of the surgeon. The material utilized for the fabric in the preferred embodiment is composed of synthetic material, parts bio-absorbable and parts non-bioabsorbable. When used in this patent, the term “absorbable” incorporates “bio-absorbable” and “bio-degradable” and means the composition of the material is broken down or assimilated by the human body and there is no stiffness nor significant tensile strength remaining in the material. The final disposition of the material may or may not result in it being ultimately flushed from the body's tissue. The fabric may also be composed of threads which themselves are partially absorbable and lose tensile strength. Other materials available include those that are totally biological in nature, e.g. collagen, skin tissue, or a combination of biological and synthetic materials. The preferred embodiment FIG. 1 has the absorbable material in a soft sponge-like consistency 72 being formed as a sheet which has intermixed within the absorbable 72 material and threads of non-absorbable 71 material which will provide support as the absorbable material dissipates and new tissue forms about the remaining non-absorbable threads. Although in FIG. 1 the non-absorbable threads are shown to run in the direction of one axis, they can be running in any direction depending upon the effect you wish to obtain. Permanent materials can be, but are not limited to, nylon, prolene, polyester, polytetra flourethylene (PTFE), silk, and biological collagen. Absorbable materials may be, but are not limited to cat gut, polyglysolic acid, trimethylene carbonate, and silk. The types of materials used for both non-absorbable material and absorbable material is constantly changing and would be known to one skilled in the art.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the clamed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed:

1. A biocompatible fabric for implanting into the body to provide support comprising:
a plurality of non-absorbable threads, and
a biocompatibility absorbable sheet material, wherein the non-absorbable threads are distributed within the absorbable material.

2. The biocompatible fabric of claim 1 wherein the non-absorbable threads have a diameter between 30 microns and one tenth of a millimeter.

3. The biocompatible fabric of claim 1 wherein the non-absorbable threads have a diameter less than 150 microns.

4. The biocompatible fabric of claim 1 wherein the sheet material is coated with a material to reduce scar tissue formation.

5. The biocompatible fabric of claim 1 wherein the sheet material is infused with a material to reduce scar tissue formation.

6. The biocompatible fabric of claim 1 wherein the sheet material is infused with a medical material.

7. The biocompatible fabric of claim 1 within the sheet material is coated with a medical material.

8. A biocompatible sheet material for implanting into a body to provide: support comprising,
an absorbable material in the form of a sheet; and
a plurality of non-absorbable threads wherein the non-absorbable threads are distributed on the exterior surface of the absorbable material.

9. The sheet material of claim 8 wherein the consistency of the absorbable sheet material is sponge-like.

10. The sheet material of claim 8 wherein the absorbable sheet material has a varying thickness.

11. A biocompatible fabric for implanting into the body to provide support comprising;
a plurality of non-absorbable threads of a diameter of less than 150 microns,
a biocompatible absorbable material coating the threads, wherein the coated threads are formed into a fabric.

12. The fabric of claim 11 wherein the absorbable material contains a medical material.
